(19) United States
(12) Patent Application Publication
(10) Pub. No.: US 2016/0037755 A1
(43) Pub. Date: Feb. 11, 2016

(54) AUTOMATED MONITORING OF RUMINANT HEALTH AND BREEDING PARAMETERS

(71) Applicant: DVM SYSTEMS, LLC, Greeley, CO (US)

(72) Inventors: Wade W. WEBSTER, Woodinville, WA (US); Jason M. WILD, Denver, CO (US)

(73) Assignee: DVM SYSTEMS, LLC, Greeley, CO (US)

(21) Appl. No.: 14/777,005

(22) PCT Filed: Mar. 17, 2014
(86) PCT No.: PCT/US2014/030343

§ 371 Date: Sep. 15, 2015

Related U.S. Application Data
(60) Provisional application No. 61/789,602, filed on Mar. 15, 2013.

(51) Int. Cl. A01K 29/00; G06F 19/00

(52) U.S. Cl. CPC A01K 29/005; G06F 19/3418; G06F 19/322; A61B 2503/40; A61B 2562/0271; A61B 2010/0019

(57) ABSTRACT

An automated system and method for obtaining early detection of biological changes or events by assessing core body temperatures that precede the events within individual animals in a production herd. The system and method may monitor the animals, assess the data acquired with a variation from a diurnally compliant baseline in the selection of or use of data monitored, and provide a timely communication to owners and operators as deemed appropriate. An assessment may establish variations from the baseline, compensate for ambient conditions or identify patterns of variation, that anticipate estrus, ovulation, illness, calving or other biological events throughout the herd population. The assessment may include signal processing techniques that substitute for baseline establishment, or be used in combination with baseline variation assessment.

### CROSS-REFERENCE TO RELATED APPLICATIONS

This application claims the benefit of U.S. provisional application Ser. No. 61/789,602 filed Mar. 15, 2013, the disclosure of which is hereby incorporated in its entirety by reference herein.

### TECHNICAL FIELD

The present invention relates to production monitoring of biological events in animals with communication of sensor signals from each animal, for computer processing of detected data incorporating processing of algorithms to associate patterns or variations of core temperature within the animal as indicators of biologic events or conditions of the animal that improve breeding, calving, animal health and production of milk.

### BACKGROUND

Reproductive performance of the dairy cow has decreased dramatically over the last 25 to 50 years on dairy farms, and the lack of estrus detection or inaccurate detection are major reasons for increased numbers of days open for the average cow. The majority of cows culled from dairy herds are due to poor reproduction. Poor reproductive performance means decreased total milk production and one less calf over the lifetime of the cow according to known reports.

The preventable economic losses from conventional failures in health management dairy programs involve both short and long term effects on milk yield and components, on disease incidence and severity, on subsequent reproductive performance, and the associated labor and treatment costs. Dairy cattle death losses in particular, are an economic disaster and represent very substantial problems with animal well-being (For example as reported by McConnel in 2008). Records show that over the past two decades, dairy cattle mortality rates have been increasing. Increasing dairy farm sizes have reduced the time dairy producers can devote to individual monitoring of cows. Research has identified various management practices or characteristics of dairy operations across the country and determined that dairies lack the personnel with training and experience to identify early stages of disease and apply timely and appropriate treatment (Garry 2002). Current herd monitoring for mastitis, as an example, remains sub-optimal or ineffective for the treatment of most intramammary infections. Therefore, improving health monitoring systems to detect problems as accurately and rapidly as possible may improve milk production and reduce costs.

The fresh cow program uses labor-intensive manual rectal temperature monitoring the first 7 to 10 days postpartum (Upham, 1996). Supportive therapy is based on how the cow was classified according to the presence of fever and if it appears sick. This protocol has been further developed by Aalseth with the understanding that the fresh cow is often encumbered with more than one disease (Aalseth in 2002).

Currently, reported estimates establish that approximately seventy-five percent of disease in the dairy cow occur within the first 30 days in milk (DIM) of each lactation. The economic impact of disease is further realized in understanding that a cow must reproduce and provide adequate milk production well into the second lactation in order to break even on feed costs. For example, Aalseth has reported that there is a $6 return for every dollar invested in a fresh cow program that includes rectal temperature for the first 10 DIM.

Alterations in body core temperature remain a concomitant of the disease state. Fever, by definition, is a well-controlled response of the immune system that resets the resting body core temperature above the normal body temperature. Fever is also the cardinal vital sign for infection and is present before other clinical signs or symptoms of an illness. It is estimated from previous unpublished work that fever typically precedes disease states by 2-3 days ahead of clinical signs or symptoms including a decrease in milk production (see “Temporal Comparison of Temperature, Somatic Cell Count and Milk Production in E. coli Mastitis”). Numerous studies in fresh cows that followed temperature elevations for more than 48 hours have demonstrated fever to be a reliable indication of infectious disease that indicates an intervention for treatment is warranted. For all cases, a temperature elevation of 48 hours indicated a serious change in vital signs and require an immediate evaluation as to its cause. (Benzaquen et al., 2006; Miner 2002; Upham, 1996; Risco Monitoring Postpartum Health in Dairy Cows; Dobberstein, Colorado State University, Fort Collins, Colo.; Stevens et al., 1995; Dinsmore et al., 1996; Smith et al., 1998; Kristula et al., 2001; Zhou et al., 2001; Drillich et al., 2001, 2003; Risco and Hernandez, 2003; Chenault et al., 2004). There are other instances where a fever may also appear with certain drug reactions, vaccinations, parturition, estrus or cancer. Previously, an induced mastitis in an untreated animal saw a reduction of milk production dropping several pounds within ten days and not rising beyond about seven pounds less over a week and affects future reproductive success.

Known methods of monitoring individual animal health and biological events of ruminant animals have not been broadly adapted for use in a production operation primarily due to cost. In line analysis of milk has technical limitations in terms of sensitivity and specificity for production, disease and purposes of breeding. Activity based monitoring for changes in behavior for purposes of breeding has difficulties in field application due to animals in crowded environments, on concrete surfaces and time spent in milking parlors.

A previously known analysis of animal health is to analyze blood to detect the level of progesterone and pregnancy which requires the manual labor of blood sampling. Blood samples must also be physically taken and sent to laboratories for analysis, a process not conducive to monitoring each animal on a daily basis with a large herd.

Another form of analysis is ultrasound imaging that is difficult, time consuming and costly to apply throughout the herd. Presently, ultrasound evaluation for detecting pregnancy must be performed at 28 to 30 days, which is well outside the 21-day estrus cycle. The realities of this problem are that “peak milk production” is a window of time soon lost after calving and that cows can become profitable again only if the breeding is successful to re-introduce “peak milk production” (see “Individual Cow Lactation Curve”. In addition, breeding events such as ovulation or parturition (calving), may not be recognizable or identified at an early stage in the breeding process by such testing throughout a large population. Even with the known data gathering techniques, /biological cycle and health affects production of the animal.

After calving, the animal starts lactating. A voluntary waiting period (VWP) of about 45-80 days may occur before attempts to breed may be initiated. Earlier attempts to breed may be initiated, but generally have been found to negatively affect milk production and chances of impregnation. Any delays in pregnancy result in costly Days Open during which cows must be fed and treated, and the subsequent lactation cycle is delayed.

### SUMMARY

The present invention overcomes the above disadvantages and known manual procedures for monitoring temperatures by providing an automated system and method for obtaining early detection of core body temperatures that precede or accompany biological changes or events within individual animals in a production herd. The system and method may monitor the animals, assess the data acquired, and provide a timely communication to owners and operators as deemed appropriate. An assessment may establish a baseline temperature for each animal, and monitor the variations from the baseline, or patterns of variation, that identify or anticipate estrus, ovulation, illness, calving or other biological events throughout the herd population. The assessment may include signal processing techniques that substitute for baseline establishment, or be used in combination with baseline variation assessment.

In a preferred embodiment, monitoring and detection may be facilitated by bolus sensors, and/or other sensors, that are conveniently stored in ruminant animals, such as dairy or beef cattle or other mammals. For example, in the reticulum, a bolus may provide radio-frequency signals of temperature readings sensed at the bolus to monitoring stations near which the animals may be regularly or routinely displaced. The bolus is particularly identified so that individual animals, and their temperature readings, are distinguished within the herd and accumulated. Computer processing and software may be employed to accumulate and store the data, read or assess the bolus data sensed, and may employ algorithms that establish a baseline temperature for the individual animal. The baseline may be filtered from noise such as water content in the reticulum (water drinking), stress, illness, breeding or calving cycle, or ambient temperature that may affect the sensed temperature. The system and method may determine that the readings represent a temperature difference, or a pattern of variation, within the animal that identifies an illness condition, an estrus period, an ovulation event, imminence of parturition, or other biological event. The system and method may then generate an alert to be delivered as required by the herd-owner's needs, such as a graphic user interface, trigger a sort gate, animal marking device or other warning system.

Embodiments of the present invention may provide temperature monitoring that promptly and accurately describes health events. Such improvements should result in improvement of health and profitability for a dairy operation. Marking or selection systems which make a mark on the cow or automatically select an animal can help speed up daily examinations. Temperature monitoring is most likely to be adopted as a component parameter in precision dairy management. Other parameters obtained such as milk weights, pH, quarter milk conductivity, animal movement and body scoring may objectively identify individual animal health. The embodiments may assess the feasibility of a health monitoring system deployment into a large dairy based upon the opportunity afforded from temperature monitoring.

### BRIEF DESCRIPTION OF THE DRAWING

The present invention will be better understood with reference to the accompanying drawing figures, in which like reference characters refer to like parts throughout the views, and in which:

FIG. 1 is a schematic view of a monitoring system for collecting and analyzing sensor data as animal data records for biologically related health assessments;

FIG. 2 is a flow chart of an algorithm processed at one or more locations in the schematic diagram of FIG. 1;

FIG. 3 is a flowchart of an algorithm processed at one or more locations in the schematic diagram of FIG. 1;

FIG. 4 is a flow chart of an algorithm processed at one or more locations in the schematic diagram of FIG. 1;

FIG. 5 is a flow chart algorithm processed at one or more locations in the schematic diagram of FIG. 1;

FIG. 6 is a graphic view of a timeline for a bovine reproduction cycle demonstrating detection of estrus and identification of an optimal time period for insemination;

FIG. 7 is a graphic view of a bovine estrous cycle, breeding cycle events and predicting ovulation with the system of FIG. 1; and

FIG. 8 is a graphic representation of temperature pattern recognition related to estrous and ovulation overlying results of other standards for testing for these biological events.

### DETAILED DESCRIPTION

In the preferred embodiment, identification of the breeding events of estrus and ovulation are identified by monitoring core body temperature decreases and increases, while selectively excluding one or more noise factors. Factors, such as water drinking effect, stress, illness and ambient temperature may have an impact upon the bolus readings and may be compensated for or removed to establish a temperature baseline or when otherwise assessing the readings acquired.

Assessing may include establishing a baseline and monitoring temperature variations or patterns of variations from the baseline while identifying correlations developed as representative of imminent biological events. Alternatively, assessing may be subjecting the readings to signal processing that generate data correlated to the biological events. Baseline comparison, pattern detection, correlation quantification, data translation and convolution, or other analysis, may be used to assess readings to identify patterns or temperature differences correlated to events. A selectively defined diurnal baseline for each animal, or a selected grouping within the production herd, may be used to uniquely assess each animal or selected grouping of the herd. These unique aspects of the preferred embodiment may be embodied in the intelligence incorporated in the DVM System's TempTrack® and TempTrack® PLUS software algorithms, that create accurate assessments. Individual animal temperature baselines may be established to identify breeding and illness events. These algorithms may incorporate filtering or analysis of noise introduced to readings by water ingestion, ambient temperature, illness, stress, and extraneous temperature effects.

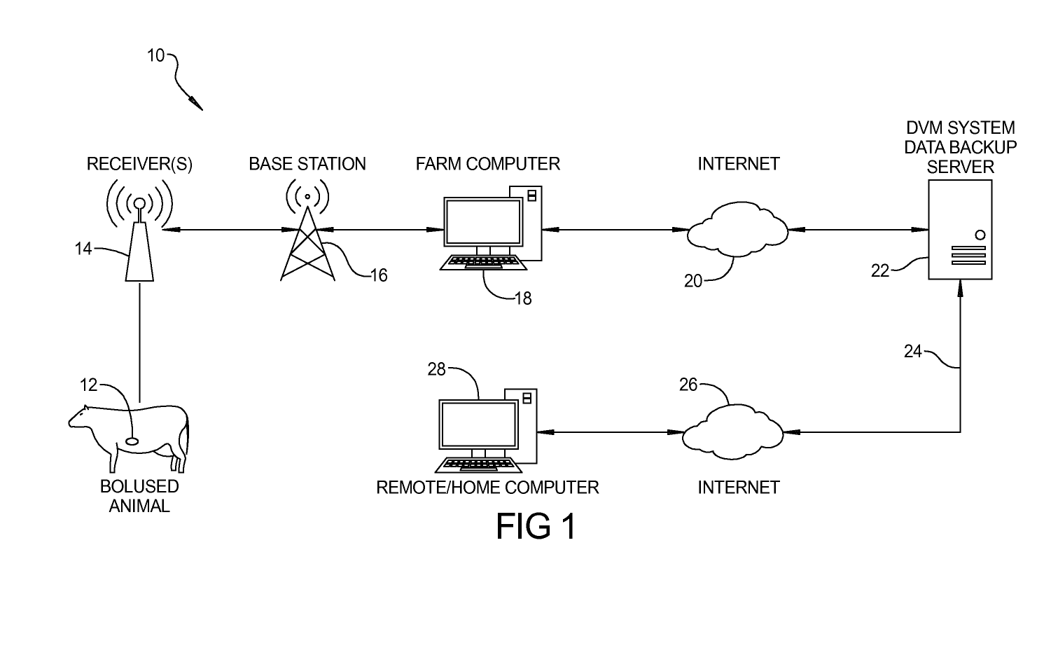

As shown in FIG. 1, an RFID embodiment of the DVM Systems Ruminant Animal ID and Temperature Monitoring System 10 may comprise three hardware components. These components are the Bolus 12, Receiver 14 and Base Station 16. In addition, there is a personal computer 18 with a software package that enables storing and displaying animal ID, Temperature and Time Stamp, animal health and breeding information, and analysis of these data that may provide early detection of biological events that may be used to generate alerts to managers or workers interacting with the herd. The software manual provides a description of typical equipment used to capture animal core body temperatures as outlined in the following DVM Systems Product Specification document for a personal computer download. TempTrack® software, version 3.4 (Jan. 10, 2013) may operate with a remote cloud or server 22 connection for data processing on site or remotely via Internet connection 20 to system server 22. Additional communications may be utilized as shown at 24, for veterinary or other management processing as at internet channel 24 and computer 28.

However, earlier versions of DVM Systems' copyrighted software also captures, tracks, analyzes and, textually and graphically displays information relative to identification and treatment of illness, health maintenance, herd management and animal reproduction improvement. The TempTrack® software supports either active or passive boluses 12 and may support other equipment. The software transfers captured, imported, and analyzed data also made available to the secure remote server 22 for backup and other purposes. The software integrates and interfaces with, imports from, and exports to, other animal management software packages that may be utilized at the farm, its computer or its server. Software created by DVM Systems, it may interact with other entities' software, to enhance the software's data analysis capabilities and the user interactions.

Optional versions available may include modules such as TempTrack® end user software for those who manage day-to-day animal operations such as producers and breeders; and TempTrack® Remote™ developed for remote access to farm data by producers, veterinarians or others who require access while away from the system location.

DVM Systems' TempTrack® copyrighted software provides automatic Animal Health Alerts. DVM Systems' temperature monitoring, temperature baseline calculations and medical data are displayed for each animal. Actual data or colored icons representative of the health of an animal is shown on a display.

Visible screen displays may utilize shaped zones or graphic lines may indicate acceptable health or warning ranges of temperatures. Temperature points may be shown within or outside zones. Health alerts may be shown as symbols and icons such as flag icons, for follow-up to alerts and may be color coded to distinguish high temperature alerts, low temperature points, and normal alerts. Icons on colored coded alerts support use by color sight challenged users. DVM Systems' TempTrack® software uses proprietary algorithms to display temperature monitoring, temperature baseline calculations, medical data, and alerts for each animal. DVM Systems' TempTrack® software also may display historical temperature data compiled as well as medical, milk, lab and reproduction data from most major dairy management software interfaced as inputs from that herd management.

TempTrack® Software Systems Requirements may include a desktop or laptop computer (can be supplied by DVM Systems) running Windows XP, Windows Vista, or Windows 7. Specifications vary with herd size. The recommended system for herd size over 500 head was an Intel i7 processor, 4 GB memory, 1 TB HD, monitor, keyboard, mouse, and internet connectivity. The system is compatible with most major herd management software packages including, DairyComp 305, Dairy Plan, DHI Plus, Easy Dairy, LIC, PC Dart and others.

The TempTrack® software may reside on same computer as facility's herd management software to facilitate integration with all applicable health and reproduction data and eliminate need for duplication or manual data transfer process. A high speed internet connection is highly recommended which helps facilitate the TempTrack® included capabilities of automatic offsite secure data backup, remote service and support, remote access and download software upgrades.

Active system sensors may come from other systems, sensors and tests such as somatic cell count, milk quality or other inputs. An exemplary active temperature RFID Bolus features accurately and automatically reading data and/or sensor inputs and transmits individual animal core body temperature, over a predetermined time period. Data logs accumulate a predetermined number of readings, for example, the last 12 readings.

Active sensors may eliminate need for animals to pass through stationary panels as in a passive system. These sensors read temperature anytime the animal is within a distance, for example, up to 300 feet (91.4 m) of a receiver 14 providing easy coverage of hospital, maternity and dry pens, or pastures.

The active system can isolate or identify a specific animal using the TempTrack® software or an RFID ear tag through integration with sort gates, parlor monitor, parlor voice annunciation or by using a hand held locator. It also provides tamper proof identification; uses FDA approved materials, contents sonically welded into FDA approved plastic casing; has expected battery life of 5 to 7 years in reticulum with hourly readings; may lower maintenance (none required); and permits storage temperature from 0° F. to 200° F. (−17.8° C. to 93.3° C.). Additional useful Bolus specifications include temperature measurement range of 87° F. to 112.5° F. (30.6° C. to 44.7° C.) (can be modified by special order); temperature resolution of 0.1° F. (0.055° C.); temperature accuracy of +/−0.5° F. (0.275° C.) (can be calibrated to a higher level of accuracy); temperature repeatability of +/−0.056° F. (0.031° C.); reading frequency hourly (optional factory programming between 1 to 60 minute intervals); Bolus: Length: 3¼″ (83 mm); Diameter: Top: 1¼″ (32 mm); Bottom: 1¼″ (31 mm); Bolus retention >99%; and data logging capability (most recent 12 readings).

An exemplary receiver in an active system may include electrical requirement: 12 V DC, 120 or 240 VAC (50 or 60 Hz) or other source; a coverage distance from bolus to receiver distance up to 300 ft. (91.4 m), A Yagi directional antenna, a beam width: (vertical orientation): 120° (horizontal orientation): 90°; and receivers to outdoor weatherproof base station up to 1 mile (1.6 km) clear line of site (subject to local conditions), omni-directional antenna; and 1.2 miles (1.9 km) directional antenna. Optional: upgrades permit up to 5 miles (8 km) clear line of site (subject to local conditions) with optional high gain omni-directional antenna or directional antenna. Receivers distance to indoor base station may be located up to 1.2 miles (1.9 km) with directional antenna.

A base station (active sensor system) may be active or passive depending upon specific activity and data accessed. An exemplary active Outdoor Weatherproof Base Station (NEMA 4, IP66) may have an electrical requirement: 12V DC 120 or 240 VAC (60 or 50 Hz); a distance from base station to PC: up to 300 ft.(91.4 m) maximum; a connection: Cat5 with serial (base station) to serial to USB adapter (personal computer) or Internet Protocol (IP) based connection, and a base station antenna may be omni-directional or directional.

An exemplary Indoor Base Station may operate with an electrical requirement: 120 or 240 VAC (60 or 50 Hz); a distance from base station to PC: Standard 5 ft. (1.52 m); and an optional up to 300 ft.(91.4 m) maximum (Cat5 connection). In addition, the connection used may be standard. For example, direct connection from indoor base station to DB9 serial connector on PC or through use of serial to USB adapter. The connection may also permit optional Cat5 connection with serial (base station) to serial to USB (PC) adapter or Internet Protocol (IP) based connection, or an exemplary solar panel may be optional for active system receivers and basestations. Such panel may have power specifications of system power draw: 100 ma, standard battery 12 VDC 20 Ah (approximately 200 hr. charge). Optional battery configurations may be available for unique requirements, and may provide optimal panel charge level: 21 v.

An exemplary passive system used may use a passive temperature RFID bolus sensor or other sensor. Such example may accurately and automatically read core body temperature or other inputs and transmit information every time animal passes through reader panels, for example, a milking parlor entrance/exit or along cow path. This bolus may isolate or identify a specific animal using the bolus identification through integration with sort gates, parlor monitor, parlor voice annunciation or by using a hand held locator. This Bolus may have tamper proof identification and employ FDA approved materials.

An exemplary passive temperature RFID bolus may have bolus specifications such as a length: 3¾″ (95 mm); a diameter: top ¾″ (20 mm), bottom ⅞″ (23 mm); FDA approved materials, sonically welded bolus; a bolus can optionally include a magnet for protection against hardware disease and to aid bolus collection at slaughter; a minimum expected life of 5 to 7 years in reticulum and is re-usable; a storage temperature from 0° F. to 125° F.; a temperature measurement range from 98° F. to 108° F.; a temperature resolution: 0.1° F. (with reader); a temperature accuracy from +/−0.6° F. (at 15 inch read range); and a bolus retention >99%.

An exemplary passive reader panel includes the panels which are made of rugged PFTE plastic housing enclosures. The two panels are mounted directly across from one another such that animals must pass between them single file. The sensor may be designed and installed in multi-panel configurations to minimize animal flow congestion. Such a panel physical description may include dimensions of each panel: 27″ H×23¾″ W×2″D; a panel designed to be mounted to one or two non-metallic posts. Such a sensor can be in a maximum panel cable distance to power supply of 20 feet. An operating environment where temperature may be −40 to 125° F.; Storage −40 to 125° F.; humidity may be 0-90% RH non-condensing; and panels will survive water spray and farm chemicals. Such sensors may have panel performance of read percentage: 98% or better when properly mounted and tuned. All metal loops in immediate proximity to front and backs of panels must be eliminated. Temperature resolution may be 0.1° F. (with System Software); and reader range meets read percentage requirement when cows pass through panel reader spacing of 32 inches at a speed not to exceed 2 feet per second.

An exemplary passive system power supply unit may have physical qualities of a panel reader power supply enclosure UL 50 listed and CSA certified NEMA/EEMAC Type 1 & 3R, rated for indoor and outdoor use, falling liquids and light splashing. A hinge-cover enclosure has a galvanized steel continuous hinge, and external mounting plates. A 16-ga. steel enclosure has a drip-shield top and knockouts on the bottom to keep rain and moisture away from components and may be dimensioned 12.06″ H×10.00″ W×6.13″ D.

The power unit may have electrical/communication characteristics of a regulated power supply, low voltage circuit breaker, noise suppressing EMI filter; maximum input 110 VAC (60 Hz) at 2 amps or 220 VAC (50 Hz) at 1 amp single phase; a maximum output 24V, 3.6 amps; and a maximum length of panel cable and power supply of 20 feet. The electrical connections may include a terminal block for electric power supply connection.

The communication modules may include Ethernet hardwire connection via CAT5 cable up to 300 feet or Wi-Fi for distances greater than 300 feet, but typically not more than 1320 feet; and a recommendation that power supply be connected to a Smart Uninterruptable Power Supply (UPS) to protect electronics. Another recommendation is that electrical, CAT5 or CAT6 Ethernet, and panel cables must all be placed in conduit, and electrical and all power supply unit cables should be separated by a distance of at least 6″.

The embodiment may include other DVM systems integrated hardware/software system solutions. DVM has developed software and firmware which is integrated with a variety of other hardware and devices and systems to identify, locate, sort and isolate animals including a hand held locator. DVM Systems' Hand Held Locator (HHL) provides an alternative means to locate and identify animals with health or breeding alerts or which otherwise need to be located for treatment, vaccinations, etc. Some typical remote reading applications include hospital areas, maternity pens, remote holding pens and pastures. RFID ear tags may provide useful communications and data and are compatible with data and sensors from any existing system previously installed. A device may identify animals from a pre-loaded list and does not take a real-time bolus reading.

An embodiment may utilize sort gate activation which DVM Systems has integrated with many sort gate manufacturers resulting in a unique ability to sort your animals based on most TempTrack® and other parameters such as dry cows, by pen number, for vaccinations, for breeding, for treatment, etc.

An embodiment may have visual monitors display DVM TempTrack® health alerts, ear tag numbers, temperatures, follow-up or other animal management data (i.e., lost cows, etc.) to your parlor, hospital or other staff. DVM Systems uses the highest quality industrial monitors. A 17″ model may have specifications such as a 17″ active-matrix LCD display, 1280×1024 resolution; a black powder-coated carbon steel or stainless steel faceplate; a NEMA/UL Type 12/4 or 12/4/4X; an analog resistive touch screen, tempered safety glass, or acrylic protective window options; 52.5 mm (2.07″) deep behind panel; a rack mount option; a NEMA 4/4X rated for wash-down applications; high temperature, shock and vibration specs; a power input—120/240 VAC, 1.5/0.75 A, 60/50 Hz; field MTBFs greater than 250,000 hours; 50,000 hour backlight brightness half-life; and may be tested to IEC Reliability Standards. Additional recommendations include: UL 60950 3rd Edition/cUL recognized component (File No. E212889); UL_{—}508 A Listed (File No. E318630); FCC Class A; CE; RoHS; WEEE (Registration No. WEE/DJ1859ZX for UK only); IEC 60721-3; UL 50E (File No. E318630)/UL Rated for Class I, II, III.

An embodiment may interface with voice/annunciation/sound systems—announce DVM TempTrack® Health Alerts, ear tag numbers, temperatures, follow-up or other animal management data (i.e., lost cows, etc.) to your parlor, hospital or other staff. They may also interface with visual tagging/marking systems, and automate animal identification of animals with DVM TempTrack® health alerts, specified ear tags, animals in need of pregnancy checks, or vaccinations or other animal care. A DeLaval Cell Counter may be used in conjunction with DVM Systems' animal health alerts to assist in diagnosing mastitis and level of infection by measuring somatic cell count level.

TempTrack® PLUS may add estrus detection and ovulation prediction capabilities to sensing technology. These functions may be used in conjunction with the active bolus product. Amplification may be used to extend read range between active bolus and receiver.

DVM Systems, LLC End User Software named DVM Systems TempTrack® software, DVM Systems TempTrack® PLUS software may include breeding and parturition, DVM Systems TempTrack® Remote Software for use by veterinary personnel or others remotely accessing the software data and processing and DVM Systems TempTrack® Academic software for research, data acquisition, search, and manipulation. DVM Systems' TempTrack® software captures, tracks, analyzes and, textually and graphically displays information relative to identification and treatment of illness, health maintenance, herd management and animal reproduction improvement, supporting either active or passive RFID temperature monitoring systems, other sensors, or both concurrently. Versions available include TempTrack® end user software which may be most advantageous for those who manage day-to-day animal operations such as producers and breeders. TempTrack® Remote™ was developed for remote access to farm dairy by producers, veterinarians or others who require access to data while away from the system location. TempTrack® Academic was developed for academic and research applications providing flexible analysis, reporting and exporting options. TempTrack® PLUS combines early illness detection with breeding and calving alerts. DVM Systems TempTrack® software ver. 1.1 includes a Disease Alerts Screen Display.

Another version improves user interface utility by changing the interaction with data selected. Instructional content of the user guide should be followed after completion of all of the steps contained in the DVM Software Installation Guide, for TempTrack® software. Installation can be verified by going to: Start menu\All Programs\DVM Systems\TempTrack®. Software installation is complete if the DVM Viewer opens to a reports screen showing the DVM Systems logo.

Entering Bolus ID numbers and Ear Tag ID numbers or other sensor identifiers may be handled by going to the Multi Reader Monitor software (MRM) which is accessed by going to Start\Programs\DVM\MRM. Once the MRM application is open, click on Add Bolus, add Bolus ID number and Ear Tag number, click OK. To facilitate easier association of Bolus ID numbers with Ear Tag ID numbers, the user can preload Ear Tag ID numbers. The Ear Tag ID numbers will then be accessible in a drop down list as a csv (comma separated value) file. To preload Ear Tag ID numbers, save the numbers on a spreadsheet in a column as a csv file, name the file EarTagID.csv and save the file to the MRM folder located at C:\Programs\DVM\MRM. Bolus ID numbers input may be limited to be entered into the system a limited number of times, for example, twice before they are locked out. Re-entry of a Bolus ID that has been locked out of the system requires a one-time unlock code that may be obtained by contacting DVM Systems customer service.

A Bolus ID number may be removed from the system by accessing the MRM application by going to Start\Programs\DVM\MRM. Once the MRM application is open, click on Remove Bolus, highlight the desired bolus to be removed and click OK. A user may edit a Bolus ID number or associate the Bolus ID number with a different Ear Tag ID number, access the MRM application by going to Start\Programs\DVM\MRM. Once the MRM application is open, click on Edit Bolus, change the desired information and click OK.

A TempTrack® Report Software, may have options for report viewing using the DVM Viewer. A first report option may be the Disease Alerts and a second option may be the All Animals tab.

A Disease Alerts option provides the capability to identify individual filter parameters and display choices for alerts including date range, Fahrenheit or Celsius, low and high temperature settings, Baseline calculation method and degrees over baseline. Filters may be controls for setting standards for alerts, such as a predetermined variation of temperature from a baseline, or a predetermined number of selected variations over a time period to signal an alert to be displayed. The Disease Alerts tab displays the following fields: Cow ID may be included. If used, this field will be populated with a unique identifier assigned by an individual farm; Ear Tag ID may be used; an identification number on Ear Tag ID may provide additional animal identification; Pen number may be included; DIM may be included; Lactation number may be included.

Bolus ID provides tamper proof identification as the Bolus is not ordinarily removed other than research needs require. An Alert Read Time may be displayed as date and time of most recent temperature reading. An Alert Reading may be displayed. For example, a temperature reading of most recent reading. An Alert Baseline may be displayed. An Alert Baseline may be a rolling average calculated differently based upon parameters such as the number of baseline days selected and the baseline calculation method selected. The “WindowxHour” and the “Cosine to Tenthpower” methods may be commonly used methods. The “WindowxHour” selections begin with the most recent temperature reading and combine the temperature readings within the “x” number of hours range. (i.e. “Window4 Hour” baseline method will average only those readings for the number of days selected that are within two hours before and two hours after the current reading time. (The “WindowxHour” method averages only those readings for the number of days selected that are similar to the current reading time but will sometimes produce gaps due to cows pulled from the line). Other methods use various approaches such as the “Cosine to Tenthpower” that averages all readings for the selected number of days with less weight given to those readings falling further from the current reading time but this method may result in fewer gaps when graphing results.

An Alert Difference may be the difference between the current reading and the Alert Baseline. A lowest reading may be the lowest reading used in the calculation of the baseline. A highest reading may be the highest reading used in the calculation of the baseline. Individual cow details and a corresponding graph can be displayed by double clicking on any number. The detail page may be selected to display animal data records. Animal records data may include a Cow ID. If used, a cow ID may be displayed in a field populated with a unique identifier assigned by an individual farm. An Ear Tag ID may be displayed in a field with an identification number on ear tag ID. A Bolus ID may be displayed to a field with tamper proof identification number of individual cow bolus. Fields also display readings date range, a report date range selected; a pen number for a cow's current pen number; a DSF for days since fresh; a DSB for days since bred; a LDOT#M for last day of test, milk weight; a PDOT#M for Previous day of test, milk weight; a LDOTSCC for Last date of test, Somatic Cell Count, a PDOTSCC for Previous date of test, Somatic Cell Count, a M.CNT for Mastitis count, and a LACTNO for Lactation cycle number and other customized fields as required.

A graph display may show individual cow results based upon the user's selection criteria. The “X” axis shows the date of the readings with the vertical line representing midnight or the beginning of the next day. Readings within the vertical lines are all attributed to the date shown. The “Y” axis shows the temperature level of the readings. The display may include multiple levels of color and of shading. In an embodiment, the blue shading has three different levels. The darker blue-shaded area represents one standard deviation from the baseline. The medium blue-shaded area represents two standard deviations from the baseline, while the light blue-shaded area represents three standard deviations from the baseline. Green dots may represent temperature readings that are within one standard deviation of the baseline. Red dots may represent temperature readings that are higher than three standard deviations from the baseline. Two to three red dots combined with other high risk factors such as previous mastitis cases or declining milk weights may indicate potential disease (mastitis, metritis, pneumonia, etc.) These instances require close physical observation of the cow for the following three to four days to pick up early clinical symptoms. Blue dots may represent readings that are lower than three standard deviations below the baseline. Two to three blue dots combined with other physical observations such as sluggishness, loss of appetite, lower milk weights may be used to indicate an illness and trigger an alert. A red line may represent the calculated baseline figure based upon the user's selection criteria. Hovering over a temperature dot on a displayed screen with a cursor will display that temperature point's date, time and difference from baseline. In another version of the TempTrack® software, alerts may be indicated by a star colored according to a high or low temperature alert.

The lower graph plots the same temperatures aligning each individual temperature's rolling average baseline at the “0” level and shows the difference from the baseline to each individual reading.

A Read Time field may designate date and time of reading the temperature reading for that date and time. A Standard Deviation field may designate a standard deviation of a reading. A Baseline Readings field displays readings of animal data records used to calculate assessed data baseline and their differences from the baseline. A Temperature Readings section may list all temperature readings within the selected parameters for the selected cow. The following fields may be listed: a Search Feature may display an individual cow's temperature details, selected by using the search feature located in “Disease Alerts” or “All” screens. The Ear Tag ID, Bolus ID or Cow ID may be typed into the search box and the desired cow details will appear in the main details area. An All Tab may provide a list of all bolused cows with their associated data. Detailed information on individual cows and temperature graphs can be obtained by double clicking on the Ear Tag ID, Bolus ID or Cow ID numbers on the screen displayed.

Data may be exported from your Dairy Herd Management software on a timely basis, such as daily, to ensure reporting of the most recent cow information while viewing tracked temperatures. A user may export a previously identified set of data into a custom text or data report saved to a text or data file.

For LIC Dairy Herd Management software and other similar dairy herd management software, a custom reports section in your management software may be accessed to assess data and generate alerts, reports or other display indicia. An embodiment of processing or generating outputs may export files such as Current Herd Test Results and Health Detail Form files to your DVM Folder and saved as text .txt or data.csv files. After exporting and saving these two files, access the DVM Report Viewer, Go to File\Import Dairy Data and Select LIC. DVM Viewer will prompt the user to browse for the Current_Herd_Test_Results.txt file which may be located in the DVM Folder. A wider search may be performed by entering in several digits of an ID which will result in all cow details for the IDs that meet the numbers entered. After selecting the file the DVM Viewer will then prompt you to browse for the Health_Detail_Form.txt file which may be located in the DVM Folder. When selecting the file, the following data from your LIC Dairy Herd Management software may be available for viewing fields in the DVM Systems Report Viewer.

A Medical History field may display medical history noted by farm personnel in their Dairy Herd Management software. Temperature data may be automatically populated into the DVM Systems Report Viewer throughout selected time periods on a near real time basis. The fields such as “Disease Alerts” and “All” pages may be printed by going to File\Print.

Identifying Potential Disease Cases may be performed as several consecutive or nearby temperatures outside of a parameter zone, for example, the third standard deviation or significantly above the baseline combined with other correlative data such as previous mastitis cases or reduced milk weights displayed may be assessed as indicating potential disease. High temperatures typically indicate infection or other symptoms due to mastitis, metritis, pneumonia and other illnesses. High temperatures may also be caused by a recent injection. Low temperatures can indicate ketosis (hypocalcaemia or milk fever). Low temperatures may also be caused by recent water intake of cold or cool water, although the software can adjust for these low temperatures. High and low temperatures may indicate illness before there are any apparent clinical symptoms. Therefore, temperature indications may be combined with several days of physical observations including body scoring and observance of changes in behavior such as a reduction in milk weights and/or feeding.

Modules and enhancements may add ovulation identification, disease temperature signature indicators, parturition indicators, hypocalcaemia indicators and treatment protocols. These modules may draw intelligence from research sponsored by DVM Systems at several universities as well as from data mining of our growing temperature database.

Another embodiment of user operated software may improve installing DVM Systems® Software, additional Help—DVM Systems Contact Information, Software Version, Remote Support Using TempTrack® Software may monitor conditions as are selected. Tabs for Summary, Health Alerts, Follow Ups, All Animals display Data Fields, for selecting a display for viewing a Specific Cow's Data Individual Cow Data Graphs and displaying Temperature Readings, Medical Data, Historical Notations. The graphic user interface also permits Setting Alert Parameters, viewing Follow Ups, Clearing alerts, Restoring alerts to the alerts inbox, drop down menus, for example, including File, View, Readers/Receivers, Boluses, help to permit functions such as Sorting, Searching/Scrolling, Entering, Editing, Removing and Mapping animal data records such as Bolus ID Numbers to Ear Tag ID Number, and integrating Dairy Herd Management software data into TempTrack® software.

The version may include Optional fields such as Parlor Monitor, Parlor Voice Annunciation, Sort Gate Actuation, and Hand Held Locator. Identifying potential illness cases, calving alerts and breeding alerts may also be provided. Software installation may be completed by your dealer or system installer and can be verified by going to: Start menu\All Programs\DVM Systems\DVM Viewer. If the DVM Viewer option is displayed, your DVM TempTrack® installation is complete. A shortcut icon on your desktop named “TempTrack® Viewer” or “Temp Track may be double clicked to run the TempTrack® software. The software may also be started by going to Start menu\All Programs\DVM Systems\DVM Viewer and clicking on DVM Viewer. Software installation is complete and software is operating properly if the DVM Viewer opens the screen shown below in FIG. 14.

To contact DVM Systems, email, telephone and website contact information are provided in the help section located by clicking on Help/About on the left hand top of the screen displayed. The version of DVM Systems software currently loaded on your computer can be verified by going to this location. If remote support is required, DVM may ask your permission to remotely access your computer. If remote access if necessary, you will be advised to click on Help/Remote Support to facilitate DVM technical assistance.

Selection of the “Summary” tab displays the total number of bolused cows currently active in the system (green cow icon) and the number of cows with health alerts (red cow icon) that fall within the selected parameters. Selecting the “Health Alerts” tab displays a screen with an alert displayed for all cows with temperatures falling within the selected parameters. Health Alerts can be viewed for different time periods by clicking on the drop down list located directly below the Health Alerts tab. Choices include All Animals, Alerts Inbox, Individual Day Alerts for a previous period, for example, seven days, Alerts 9/28/12 (10), or Custom Range, which allows selection of any previous day or range of days.

Typically, you would want to select and print the Alerts Inbox list that includes all cow alerts not considered previously or alerts that have not been cleared out of the Alerts Inbox from previous days midnight last night. For example, if today's date is July 24^{th} at 6:30 am, by selecting the Alerts Inbox, you may receive all alerts from 12:00 am July 23^{rd} through the current time plus any alerts that have not been cleared out of the Alerts Inbox from previous days shown in FIG. 15.

To display all alerts for all animals for all time periods, select All Animals on the Health Alerts drop down list. Health alerts may be displayed for any single day for any previous selection of days. For example, seven days, by clicking on the drop down list located directly below the Health Alerts tab and selecting your desired date. Clicking on today's date may display only those alerts that have occurred up to the current time since midnight. If selecting only the previous day's alerts (12:00 am midnight September 28th through 11:59 pm September 28th by selecting Alerts 9/28/12 (10) from the drop down list under Health Alerts, only the requested result is displayed.

Selecting a date that may not be shown on the drop down list, is enabled by clicking on the “Custom Range” link and clicking on the calendar icons to select your date or date range. To display all current alerts for cows that fall within the selected parameters, select Alerts Inbox on the drop down list. The Alerts Inbox contains the most recent alerts that have not been manually removed by the user. If an individual cow has more than one temperature alert at the time the original alert is still in the Alerts Inbox, a cow icon showing multiple cows will show for that cow.

The “Follow Ups” tab may list all follow ups that have been entered. Follow ups can be used as a reminder to examine, observe or treat a particular animal. Instructions regarding the use of the “Follow Ups” feature are described in detail below.

The “All Animals” tab may display a list of all bolused cows with their latest temperature reading. Data fields on screen may correlate to functions and term definitions as follows: An Alert is an indicator shown as a line of data or an icon on the “Health Alerts” screen used to notify a user of a cow with a sensor reading such as a temperature reading that has fallen outside the set parameters. An active alert may show a colored cow icon. Multiple icons indicate multiple alerts. Another color of cow icon indicates the alert has been resolved. A further colored cow icon used on the “All Animals” screen may indicate the latest reading for a specific cow and does not indicate whether or not there is an active alert.

A cow's baseline may be calculated based upon selected criteria determining whether sensor parameter value is within a standard or normal range for that value. A Baseline Reading represents a set of animal collected data readings or records used to calculate baseline and their difference from the baseline. A Bolus ID may be a tamper proof identification number of an individual cow's bolus. A Check Box may be a selection box on the “Health Alerts” screens used to remove an individual alert once the animal has been examined or the alert is no longer required. Once one or more of the check boxes have been selected, buttons at the upper left of the Alerts screen will become colored, a first selected color, such as green where green has been predetermined for corresponding to a healthy cow or one not needing attention, and red corresponding to a cow requiring attention. If one or more check boxes next to Alerts are selected, the alert or alerts selected can be removed from the Alerts screen by clicking on an appropriately colored button. The alert will be removed from the “Health Alerts” screen but will permanently remain on that individual day's health alerts (i.e. “Alerts 12/7/12) for the date when it first appeared, however, the cow icon will now be colored the appropriate color such as green. A Difference Field may display a difference between reading of an animal data record and baseline temperature or other sensor animal data record.

A Follow Up indicator implements the “Follow Ups” feature that enables notation for subsequent action on a particular reading with a follow-up date and optional note, of action for physical examine or other action with a particular cow on a specific date. Last Read Time identifies a date of the most recent sensor readings, Last Reading is a display of the most recent animal data record reported. A Next Follow Up Field refers to date of next follow up.

The Pen Number at which a cow is located may be identified, and may be imported from dairy herd management software. Read Time may be a date and time of a sensor's animal data record reading. Reading is a field showing a sensor reading for that date and time. Standard Deviation of reading from previous sensor data readings for that animal may be displayed.

To view all information being recorded as animal data records for a specific cow, click on a screen icon or double click on any Alert on any Tab. For instance, from the Health Alerts Tab drop down list, on Custom Range, alerts for a certain date were selected for display, clicking on a screen icon, or double clicking anywhere on an individual Alert's line except on Follow Up will display that individual cow's data in both graphical and numeric formats (FIG. 22).

The graphical display of an individual cow's data may include temperature data points, dates, temperature baseline and other optional information shown to the left of the graph that can be imported from your dairy herd management software. Alerts can be viewed by selecting the Health Alerts screen tab and selecting one of the four following options from the drop down box: 1) “All Animals (xx)” which includes all alerts for all animals; 2) “Alert Inbox (xx)”, which contains all active alerts that have not been cleared by clicking on the (healthy) green cow icon for a date identified (xx), which may include alerts for the past 24 hours; 3) “Alert mm/dd/yy (xx)” for a particular day in the last seven days which will show alerts for that individual day; or 4) by using the “Custom Range” which allows selection of alerts for any past date or date range.

A graph display may be viewed for individual cow results based upon the parameters selected. An “X” axis may show the date of the readings with the vertical line representing midnight or the beginning of the next day. Readings within the vertical lines are all attributed to the date shown. The number of readings displayed is determined by the number of readings captured for an individual cow during that specific day. An active RFID system may capture multiple, for example, 24 readings per cow per day and a passive RFID system may capture a reading equal to the number of times an individual cow passes by a reader panel. The “Y” axis displays the sensor (temperature) level of the readings of animal data records on the left hand side of the graph.

An optional feature, if enabled, can also display additional data such as milk weights. If this feature is enabled, for example for milk weights, the scale on the right hand side of the graph will show milk weight levels.

Individual temperature data points are represented by colored dots on displayed screens. For example, for temperature data records, the color of the dot is determined by whether a temperature point falls above, within or below the selected temperature parameters. Green dots may represent temperature readings that fall within the selected parameters. Red dots may represent temperature readings that fall above the “High Alert Temperature” setting or if temperature readings fall above the “Degrees Over Baseline Alert” setting. Blue dots may represent temperature readings that fall below the “Low Temperature Alert” setting. Sensor data is automatically populated into the DVM Viewer throughout the day on a near real time basis.

An animal's temperature baseline may be shown as a solid, bold green line. The Baseline may be calculated using an algorithm, but may be calculated differently, based upon parameters selected by the user, such as the number of baseline days and/or an alternate baseline calculation method.

With a baseline having been determined and displayed, the user may identify risks or noteworthy conditions from animal data records with as few as two same-colored dots. For example, mastitis or other high risk factors such as previous mastitis cases (M.CNT) or declining milk weights (LDOT#M) may indicate potential illness (mastitis, metritis, pneumonia, etc.). These Alerts permit early awareness for physical observation of the cow for the following 3 to 4 days to pick up early clinical symptoms. Moreover, multiple same-colored series of dots may be combined with other physical observations such as sluggishness, loss of appetite, and lower milk weights, to also indicate potential illness. Water intake during the previous 1½ to 2 hours can distort the significance of actual reading of animal data records relating to temperature. A displayed representation of a calculated baseline figure appears on screen, whether based upon the standard default setting or the user's custom selection criteria, and the effect may be filtered out in the baseline calculation. Hovering over a temperature dot with the cursor will display that temperature point's date, time, actual temperature and days in milk (DIM).

Below the Individual Cow Data Graphs that may be displayed are multiple separate tabs listing historical data and information sections which are accessed by clicking on any of the tab headings and are closed by clicking on the “X” located on screen to the far right of the headings. Medical, Milk, Lab and Reproduction Data tabs may be selected to display imported data from dairy herd management software programs and are available as an option for most major dairy herd software. A “Temperature Data” tab (See definitions below) contains all temperature readings, with alerts highlighted in red, for the selected animal under the headings of “Read Time”, “Reading”, “Baseline”, “Difference” and “Baseline Standard Deviation”.

The following displayed fields are listed in the temperature “Temperature Data” tab: tab Read Time indicating date and time of reading; tab Reading may be selected for a sensor (Temperature) reading for that date and time; tab Baseline may be selected for a baseline calculated based upon selected criteria; Difference may be selected for illustrating the difference between temp reading and baseline; Baseline Standard Deviation shows standard deviation of the reading.

A “Medical Data” tab may be selected to display individual medical health notations with their associated date of entry. When available, medical and health events can be imported from the dairy herd software program and are displayed in this section and as labels on the graph on the corresponding date of the notation.

The “Milk Data” tab may be selected to display a history of milk weights and/or milk connectivity, if available, an alternative to somatic cell count that measures physical effects upon milk upon application of an electrical charge. A “Lab Data” tab may be selected to display data from lab analyses results including fat, protein, lactose, solids, milk weight, milk volume and somatic cell count (SCC), if available. A “Reproduction Data” tab may be selected to display data and information relative to breeding such as pregnancy status, activity, action to be taken, insemination date, etc. A “Notes” tab may be selected to display notes for notations entered for this animal that have been entered into the DVM TempTrack® software. A “DVM Default” tab may be selected to display a recommended baseline calculation method. However, parameters are easily changed and can be set according to individual requirements.

For example, alert parameters may be set at the following default settings: temperature unit displays Fahrenheit temperature scale readings (Celsius optional); Baseline Method displays DVM Default baseline to process calculation of Data reading results, and Days displays (5) daily readings.

If animal data records are processed before a baseline is established, a high alert temperature may be set at 104.5° F. or 40.3° C. (if Celsius is selected); a low alert temperature may be set at 80.0° F. or 26.7° C. (if Celsius is selected). After a baseline is established, the default setting may be revised to a high alert temperature of 105.5° F. or 40.8° C. (if Celsius is selected); a low alert temperature of 80.0° F. or 26.7° C. (if Celsius is selected); or a degrees over baseline alert of 1.2° F. or 0.7° C. (if Celsius is selected).

When the animal data records reach thresholds at which an alert may be generated, default thresholds may be set as Required Alert Readings of 50%, Over Period of five hours, and Delay Until Next Alert of 24 hours. Parameter settings can be displayed or adjusted by going to the TempTrack® Health Alerts screen and clicking on a gear icon in the upper right hand of the screen. Settings can be returned to the default settings by clicking on the Default button. A “Health Alert Settings” dialog box can appear. A Health Alert Settings dialog box may display the current health alert settings in multiple, for example, three separate boxes, “before a baseline is established”, “after a baseline is established” and “thresholds”. A “before a baseline is established” box is used to identify temperature alert thresholds before enough temperature points have been captured to adequately develop a temperature baseline which may be determined over a period of time, for example, a number of days. The “Upper Limit” setting may be set at a lower temperature than the “Upper Limit” temperature setting which is used once enough data has been collected to establish a baseline. An alert triggered by the difference from a temperature and the baseline is typically a more accurate measure than an absolute “Upper Limit” temperature parameter.

Once a baseline has been established, TempTrack® enables the Health Alert settings shown in the screen display of “after a baseline is established” box. Although much of the initial assessing, including determining a baseline, or assessment through data processing are described in terms of temperature readings as animal data records.

Selecting a lower “Upper Limit”, a lower number of Degrees Over Baseline or a lower “Lower Limit” will increase the number of alerts. Increasing the baseline days will smooth out the baseline by decreasing the weight of any single reading thereby increasing the possibility of an increased number of alerts. For example, it may be useful to decrease the upper limit to 103.5 degrees, decrease the degrees over baseline to 1.2 degrees and increase the baseline days to 10 days. This may help identify more potentially at risk animals allowing the operator to decide whether to examine the animal based upon other health data such as milk weights, previous mastitis cases, high somatic cell count, high number of lactation cycles, etc. While setting the lower limit to a higher temperature setting will increase the number of alerts, it may also increase the number of low readings associated with recent water intake and therefore not be helpful in identification of more potentially at risk animals.

The “DVM Default” method display box for selection may activate an algorithm for establishing a baseline that mitigates the effect of at least one or multiple sources of noise that may distort the detection of important core temperature variations or patterns of variation. A simple average of temperature difference readings sensed at the bolus may minimally establish a baseline. However, the temperature reading at the bolus may be affected by conditions, such as the animal's drinking of water, that may be carried in the reticulum and reduce the temperature sensed by the bolus by a significant range, for example, one to two degrees. Although a baseline may be simply established, a normal average of temperature readings may not provide an operable baseline for generating alerts when temperature variations exceed a predetermined threshold due to non-health intervening conditions or when variations occur naturally, in relation to a Circadian rhythm or on a diurnal cycle. The data may be made more reliable by reducing the effects of the detectable influences, such as water content, or presence. Similarly, the ambient temperature of the animal may have an effect on the temperature reading sensed at the bolus. As a result, a DVM default may include at least one mitigating filter to compensate for a noise factor in animal data records.

Another noise reduction technique may be employed by the “x Hour Window” box selections that account for only temperatures taken within a predetermined number of hours range. The processing of the data to establish a baseline with the hour window baseline method will average only those readings for the numbers of periods, such as days, selected. Thus, if a four hour window is selected, readings that are within two hours before and two hours after the current reading time will be averaged. The absence of temperature readings within that time may introduce error, for example, if the cows are moved to a different pen not covered by the system thereby restricting reception of temperature readings. The range selection for the number of days used in the algorithm permits the adjustment to avoid ambient changes that may affect the animal such as seasonal differences and influences that may merely be short term, such as infections that may raise temperatures over a short period. The absence of readings within the day and hour windows may prevent the establishment of a baseline resistant to noise factors. In addition, time of day readings relying upon variations from temperatures taken at the same time of day may be more significant than readings taken at another time of the day in establishing a need for an alert or indication that a significant temperature change has been detected. Accordingly, greater temperature differences than previously read, but occurring at a different time of day, may not be processed in considering whether an alert condition has been encountered. When the baseline has been established, the analysis of pertinent data about variations, or patterns of variation, may be used to identify correlated or imminent biological events, and enables a predetermined alarm condition to generate an effective signaling or alert to proper authorities such as owners, managers or working personnel responsible for health care and breeding incidences.

In addition, a DVM Default algorithm may include proprietary analytical techniques as they are developed for removing noise and generating a reliable data record. The records may then help identify significant departures from a Circadian Rhythm. For example, in a system where data from the past five days is analyzed for a pattern occurring at a predetermined frequency, for example, once a day at a particular amplitude in accordance with a Circadian Rhythm, signal processing software analyzes differences within the rhythm that are illustrative of changes from previous patterns. DVM Systems' signal processing employs techniques through disclosed and developing proprietary algorithms to separate noise from natural, environmental and other sources and to identify those changes correlated with physiological changes known to indicate a health concern, a breeding treatment, calving or other event requiring action by a manager or worker. An alert is then created to notify a manager or worker about that particular event to facilitate action with the indicated animal.

The “Thresholds” box determines how and when an alert is created using the upper and lower limits, and the degrees from baseline settings. The “Required Alert Readings”, “Over Period” and “Delay Until Next Alert” settings allow fine tuning of health alerts. By selecting a percentage parameter in the “Required Alert Readings” box and a number of hours in the “Over Period” box, an alert is only created once a specific percentage of temperature points are outside of the set temperature parameters over a specific period of time. For example, the default settings of 50% and 6 hours mean that it will require 50% of the last six readings that are outside of the set temperature parameters to trigger a health alert. Additionally, a maximum number of health alerts over a specific period can be selected to prevent multiple alerts from occurring on the same cow in a short period of time. For example, by setting the “Delay Until Next Alert” box to 24 hours, only one alert per 24 hour period will be allowed. This is useful if animals with health alerts are checked once per day, for instance, in the morning so that another alert won't be received again later that same day.

There may be instances in which you want to set customer parameters for an individual cow or group of cows. This may be particularly useful if an animal has an unusually high baseline and continuously triggers alerts. To set customer alert parameters for one cow or a particular group of cows, a special function will need to be enabled. To enable the special function, click on the “All Animals” pull down selection on the “Health Alerts” tab. Select the cows on which you desire to set custom alert parameters by clicking on the box to the left of the cow icon. While holding down the Control and Alt keys on the PC keyboard, press the “a” key and release. This will display a gear icon on the upper left hand of the screen immediately to the right of the colored Alert and Healthy boxes. To the right of the gear icon will be a number that signifies the number of cows selected. Clicking on this gear icon will display a “Health Alerts Settings” dialog box. To set customized health alert settings, click on the radio button next to the statement, “Set x selected animals to the following settings”. Complete the health alert parameter settings and click on the “OK” button. To close this special feature, repeat the step: While holding down the Control and Alt keys on the PC keyboard, press the “a” key and release. This special function should always be closed when finished to prevent accidental changing of health alert parameters and accidental permanent deletion of cows and their corresponding data from the system. It is recommended that this function only be used by a system administrator.

A “Follow Ups” feature enables selection of a particular cow record, whether an Alert or All Animal record, with a follow-up date and optional note if you determine that you would like to physically observe or take action with a particular cow on a specific date. Use of Follow Ups is optional. A follow up for a particular date or multiple follow ups for different dates for the same cow can be entered from the Health Alerts, Follow Ups or All Animals screens by clicking on the Follow Up white flag icon on the right side of the screen.

To enter a Follow Up, click on the Follow Up flag icon on the line for the cow desired. This will open the Follow Ups dialog box. Select the desired follow up date by either clicking on the “OK” button to select the displayed default date or by clicking on the down arrow to the right of “Date” and selecting a different date on the calendar and clicking the “OK” button. Once the OK button has been clicked, the Follow Up flag icon for the cow selected will now be colored differently, for example. The default follow up date may be tomorrow's date. All follow ups may be listed on the Follow Ups tab regardless of follow up date.

Follow Ups also pop up on the daily alert list on the previously selected date along with that day's health alerts. A colored red flag, for example, flag may be associated with the date of the “Follow Up” unless the date has passed, in which case only the colored flag may appear. To add follow ups for the same cow for subsequent days, click on the “New” button, select a date, click the “OK” button. Notes can be added to the follow up by entering a comment into the box located to the right of “Note”. Notes can be added to the cow's history record by entering a comment into the box located under “History” in the space that says “Add new history item”. Notes entered into the history record will be displayed in the “History” area on the Follow Ups box and on the History record on the “Detail” screen.

A Follow Up indication, on the health alerts or All Animals screens, can be removed by clicking on a colored, for example, red follow up flag icon which will open the follow ups dialog box. Click on the “Delete” button, then click on the “OK” button. To remove an individual follow up date on a cow with multiple follow updates, click on a colored red, for example, follow up flag icon which will open the follow ups dialog box. Highlight the desired follow up to be removed by clicking on the date, click on the “Delete” button, highlight the next follow up date to be removed, click on the “Delete” button and continue until finished, then click on the “OK” button.

In an embodiment, a Follow Ups screen permits one or more follow ups to be removed in two different ways. Removing follow ups may be performed by using the Trash Can icon. A user may select one or more of the selection boxes on the far left side of the screen for the desired follow ups. The number of follow ups selected will appear to the right of the trash can icon. Click on the Trash Can icon in the upper left hand part of the screen. A “warning” dialog box may appear that says “Are you sure you want to delete x selected follow up?”. The user may click “OK”.

To remove all follow ups, select the selection box at the top of the column and then click on the Trash Can icon. A “warning” dialog box will appear that says “Are you sure you want to delete x selected follow up?” Click “OK” to remove all follow ups including multiple follow ups on individual cows.

An alternative permits removing Follow Ups by clicking a colored red, for example, Follow Up icon. An individual follow up on the Follow Ups screen may also be removed by clicking on the red Follow Up flag icon which will open the follow ups dialog box. A user may click on the “Delete” button, then click on the “OK” button.

To remove an individual follow up date on a cow with multiple follow up dates, click on a colored, for example, red follow up flag icon which will open the follow ups dialog box. Highlight the desired follow up to be removed by clicking on the date, click on the “Delete” button and then click on the “OK” button. To remove multiple follow up dates on the same cow, click on the red follow up flag icon which will open the follow ups dialog box. Highlighting the desired follow up to be removed by clicking on the date, clicking on the “Delete” button, highlighting the next follow up date to be removed, clicking on the “Delete” button and continuing such actions until finished, then clicking on the “OK” button.

Follow up notes may be deleted from the Follow up dialog box by highlighting the note and clicking the “delete” key on a personal computer keyboard. Notes on the follow up dialog box under the history section may be deleted by clicking on the red “X” next to the note to be removed. Deleted notes may be re-displayed by clicking the “Show Deleted” box.

New health alerts and alerts from previous days may remain in the Alerts Inbox until removed. To remove an alert from the “Alerts Inbox (x)” list after a potential health issue has been resolved or no check on the particular animal(s) is desired, selecting the small box(es) to the left of a cow icon for the cow health alert(s) to be removed from the Alerts Inbox. The number of cow health alerts selected will show to the right of the box. Click on the “green” box in the upper left hand part of the screen. After clicking on the green box, the selected alert(s) may be removed from the Alerts Inbox. However, an alert will always remain on the individual day's alerts for future reference, if necessary, and may now be shown with a color coded, for example, green cow icon. Cows with otherwise color coded (red) cow icons are active alerts and remain in the Alerts Inbox.

Alerts may be restored to the Alerts Inbox by selecting, on the “Health Alerts” screen, the specific day on the pull down menu, i.e. “Alerts mm/dd/yyyy (x)”. Selecting the small selection box to the left of the green cow icon on the alert you desire to restore to the Alerts Inbox and click on the green cow box on the upper left of the screen below the alerts drop down menu. This will cause the alert to be restored to the Alerts Inbox and will change the green cow icon to red (FIG. 35).

In an active receiver embodiment, menu options are shown on TempTrack® Viewer main screen at the top of the page. Options displayed include Files that may be selected to perform functions such as Import Dairy Data that may import animal health and breeding information from selected dairy herd management software. Export Bolus Readings may be selected to export bolus readings in csv or other file format or Export Lookup Receiver Data File may be selected to export data for use with RFID ear tag with a handheld reader locator.

Import TempTrack® Data File may be selected to import a saved TempTrack® data file. Export TempTrack® Data File may be selected to export a TempTrack® data file for archiving.

Print Preview may be selected to preview print selection. Print may be selected to print selection. Exit may be selected to exit out of DVM Viewer. View may provide selection options to control display of a Temperature Unit that is desired selects Fahrenheit or Celsius measurement unit. A Column Label that selects columns to be displayed on Health Alerts, Follow Ups and All Animals screens. A Show Unmapped Boluses label displays unmapped boluses when checked, hides unmapped boluses when unchecked. A Filter label selected filters data to be displayed on Health Alerts, Follow Ups and All Animals screens. A Clear Filter returns to default display of all available data on Health Alerts, Follow Ups and All Animals screens.

Readers (Receivers) may be used by a system installer such as Manage set up readers (receivers), Start Service that controls collection of readings, Stop Service that controls collection of readings; and Restart Service that controls collection of readings. Boluses allow installers to Edit Ear Tag Mappings by entering, editing and deleting ear tag mapping to boluses, and import mapping data from a file. Help provides options such as Remote Support to allow a user to authorize DVM support person to remotely access system. Help also describes DVM software version, address, email, telephone and website information. Different versions may have minor differences in functionality.

TempTrack® Software may include functions such as sorting to allow all columns on the DVM Viewer main screen to be sorted by clicking on the heading. TempTrack® Software Functions may also include searching for any individual or groups of cow data that have been collected as animal data records. Those records may be located by entering the Cow Ear Tag number, Bolus number or other search term, into a box in the upper right hand portion of the DVM Viewer main screen next to the magnifying glass. The number or term may be cleared by clicking on a colored X or deleting the number or term. A wider search may be performed by entering in a portion of a Cow Ear Tag or Bolus number which will result in all cow details for the ID's that contain the numbers entered.

TempTrack® Software may include additional Functions such as Entering, Editing, Removing and Mapping Bolus ID Numbers to Ear Tag ID Numbers manually or automatically.

A Manual Entry Process may include opening the TempTrack® Viewer, clicking Boluses, then Edit Ear Tag Mappings. Entering a bolus ID and an ear tag ID before Clicking Add. Clicking Apply saves current entries and keeps the window open for additional entries. When finished adding bolus and ear tag numbers, clicking OK saves unsaved entries and closes the window.

Bolus ID numbers may only be entered into the system a limited number of times, such as twice, before they are locked out. Re-entry of a Bolus ID that has been locked out of the system requires a one-time unlock code obtained by contacting customer service.

An Automatic Bolus ID Entry Process may be employed. After a bolus has been read by the system for the first time, it will automatically appear in the drop down list. After herd data is imported via spreadsheet file, ear tag IDs will appear in the drop down list. From the Bolus/Ear Tag Mappings box, a user may associate ear tags to the appropriate bolus using drop down lists and clicking Add. When finished associating ear tags and boluses, click Apply and OK.

Bolus and Ear Tag IDs can be preloaded using a spreadsheet file program such as a Microsoft Excel file. Bolus and Ear Tag IDs can be preloaded together or either individually without preloading the other.

To preload both the Ear Tag and Bolus IDs together, a user may follow a series of steps starting with creating a spreadsheet file with one column, name and save the file, noting the location for future use. In the first column, enter or copy the Ear Tag ID number followed by a comma, in a comma-delimited file, followed by the corresponding Bolus ID number using a full ten digit number Bolus ID number including zeroes. Continue populating successive lines using the same format. When finished entering all data, save this file. In the TempTrack® software program, click the Boluses menu, click on Import Ear Tag Mappings, browse to the location of your saved data file, highlight the file and click Open.

To preload only Ear Tag IDs or only Bolus IDs, follow a similar series of steps. Create a spreadsheet file with one column, name and save the file noting the location for future use. Any standard comma delimited file may be substituted. In the first column, enter or copy the Ear Tag ID number or the Bolus ID number using the full ten digit Bolus ID number including zeroes. Continue populating successive lines using the same format. When finished entering all data, save file. In the TempTrack® software program, click the Boluses menu, click on Import Ear Tag Mappings, browse to the location of your saved data file, highlight the file and click Open.

Editing, Changing, Removing Bolus ID or Ear Tag Numbers may also be performed. To edit/change an Ear Tag ID in an existing previously mapped Bolus ID and Ear Tag ID, from within the TempTrack Viewer, click Boluses and then click Edit Ear Tag Mappings. The Bolus/Ear Tag Mappings dialogue box will appear. Highlight the Ear Tag ID that you are changing, enter in the new Ear Tag ID, click on another item within the box. Using the same process, continue until all desired Ear Tag IDs have been changed. When Ear Tag changes are completed, click Apply, click OK, close and re-open TempTrack® software to complete change process. All data associated with the bolus number from the old Ear Tag ID will be retained.

To remove an existing bolus/ear tag association, click the red colored “X” to the right of the ear tag you would like to remove (FIG. 12-9.4). To change an existing bolus/ear tag association, click the red “X” to the right of the ear tag you would like to remove, locate the bolus number in the blank window in the lower left of the Bolus/Ear Tag Mappings window below the Bolus ID column by clicking on the down arrow to show the drop down box and highlight the desired bolus number. Then, either click the down arrow on the blank window in the lower right of the Bolus/Ear Tag Mappings window below the Ear Tag ID column, highlight the desired ear tag number and click “OK”, or enter the desired ear tag number and click “OK”.

The system embodiments provide important value to herd owners and managers as core body temperatures may indicate illness before there are any apparent clinical symptoms. Therefore, temperature indications may be combined with several days of physical observations including body scoring and observance of changes in behavior such as a reduction in milk weights and/or feeding, ordinarily, relied upon by owners, veterinarians and managers. The Follow Up feature may be very useful to keep tabs on an animal with high temperatures that does not show any immediate clinical symptoms.

DVM Systems Temperature Monitoring Network Diagram shown in FIG. 1 may also be representative of an active RFID system.

A method or a system for monitoring health of at least one mammal comprises detecting diurnal temperatures with a bolus carried in said mammal. A method step may be recording the detected data for individual animals of a herd by identifying animal data records that may include linking a sensed temperature with an animal identification and a time identification. A method step may be assessing significant sensor signal variations or patterns, or by establishing variations or patterns from a baseline of the recorded readings of individual animals of the herd by computer processing algorithms. The algorithm may eliminate noise factors affecting the readings and monitoring of the sensors by selecting or identifying temperature differences, or patterns of variation, indicative of biological conditions. A method step may be monitoring variations from said baseline above or below a threshold level. A method step may be assessing significant temperature variations or patterns by signal processing techniques, and monitoring or identifying particular patterns with or without use of a baseline using any one or more methods of identifying thresholds of variations, or patterns, or both, in combination.

Assessing may include one or more methods of statistical analysis, pattern recognition or other signal processing techniques. A method step may be initiating an alert corresponding to a monitored threshold variation. The monitoring may comprise identifying a threshold and/or a pattern of animal data records as a condition related to a function of breeding. The monitoring may comprise identifying a temperature variation threshold and/or a pattern of a temperature variation as a condition related to a function of illness. The monitoring may comprise identifying a threshold variation and/or a threshold pattern as a condition related to a function of ovulation or other biologic or breeding event.

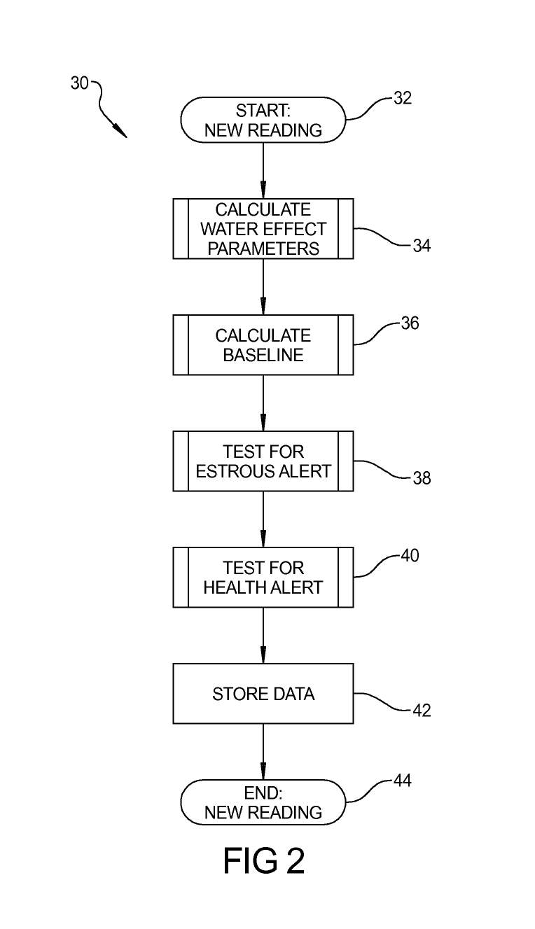

As shown in FIG. 2, the assessments may be performed as algorithms as shown at 30. The algorithm receives a reading of animal data records at 32. The assessment may include filters. A filter 34 may calculate water effect parameter, such as a selected number of readings over a specified time period, and determine whether to use, not use or compensate for a particular reading. Once the use has been determined, the assessment may proceed to calculate a baseline 36 for gauging subsequent readings. As described in this specification, alternative processing may be employed at 34, 36, 38 and 40 without departing from the present invention.

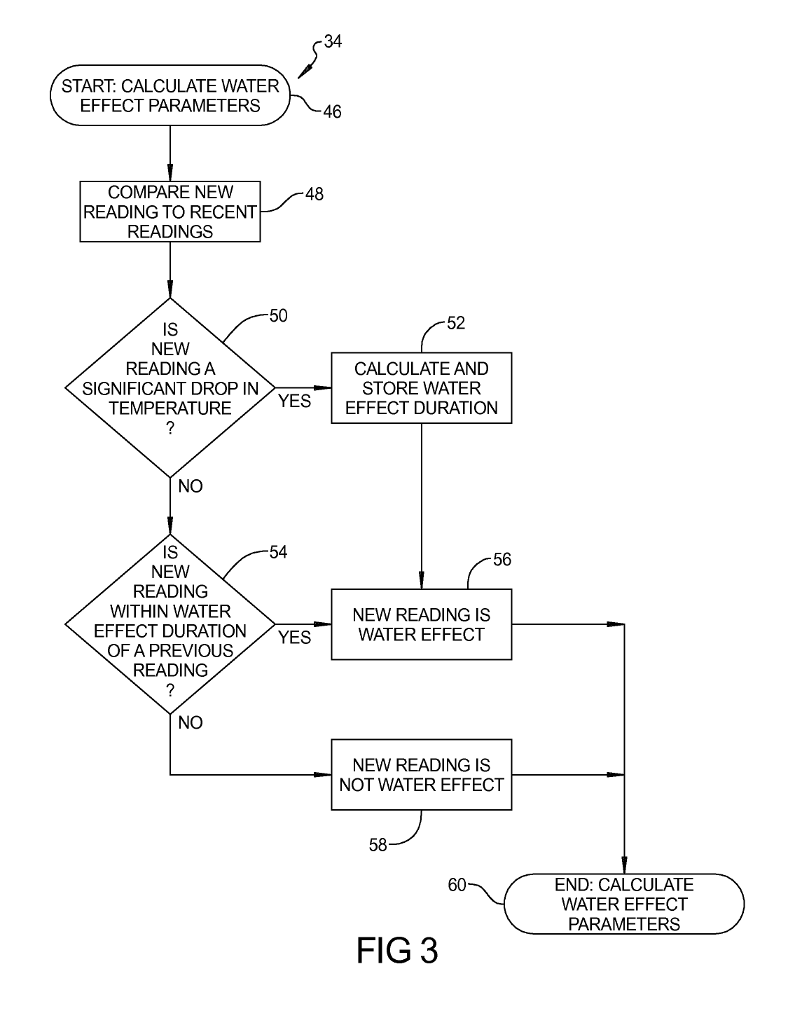

As best shown in FIG. 3, one of the ways to take water effect into account is before the determination of a baseline. In order to qualify animal data records for use in calculating a baseline, a first qualifier at 48 may be to compare a new reading with past readings, and then determine whether a threshold is reached by the comparison at 50. If the threshold is not met but the reading is not within the water effect duration of a previous reading, the new reading may be determined to not be a water effect reading. Otherwise, the reading at 52 or 56 is calculated or weighted to a water effect value.

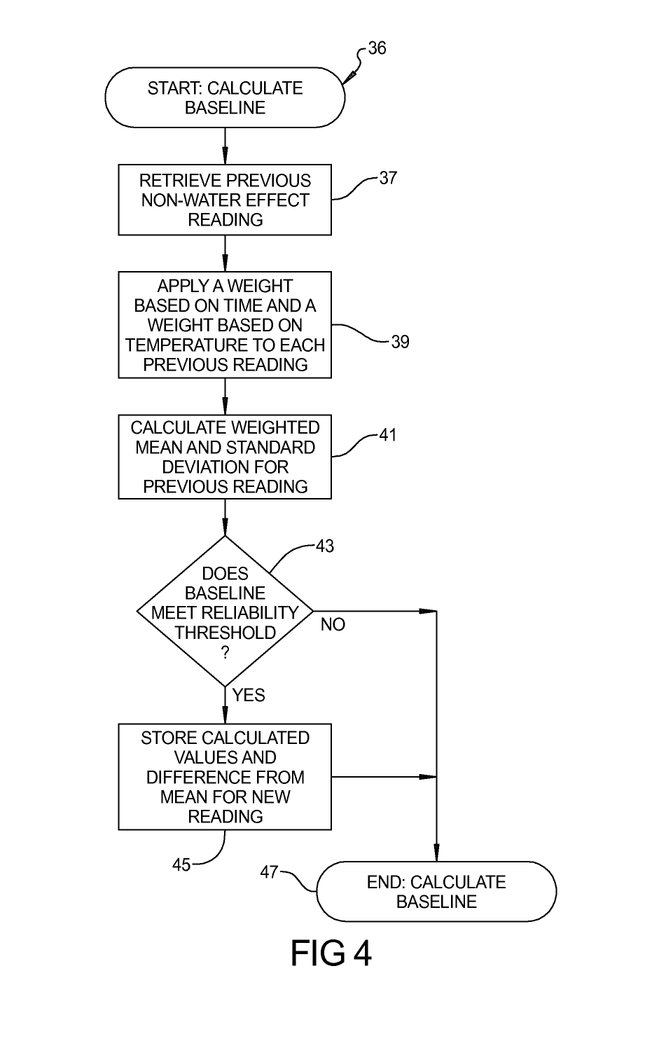

As shown at FIG. 4, a baseline may be set without recent water effect readings being used in this determination, although other methods or other compensation for use of the reading may be added. The processing 36 shows application of a weight to each non-water effect reading based on a time and a weight based on temperature at 39. Then, the mean of the weights and standard deviation of the reading at 41 may be used to compare the results with a threshold for reliability at 43. The reliable values are stored for processing along with the difference from the mean for the reliable reading at 45.

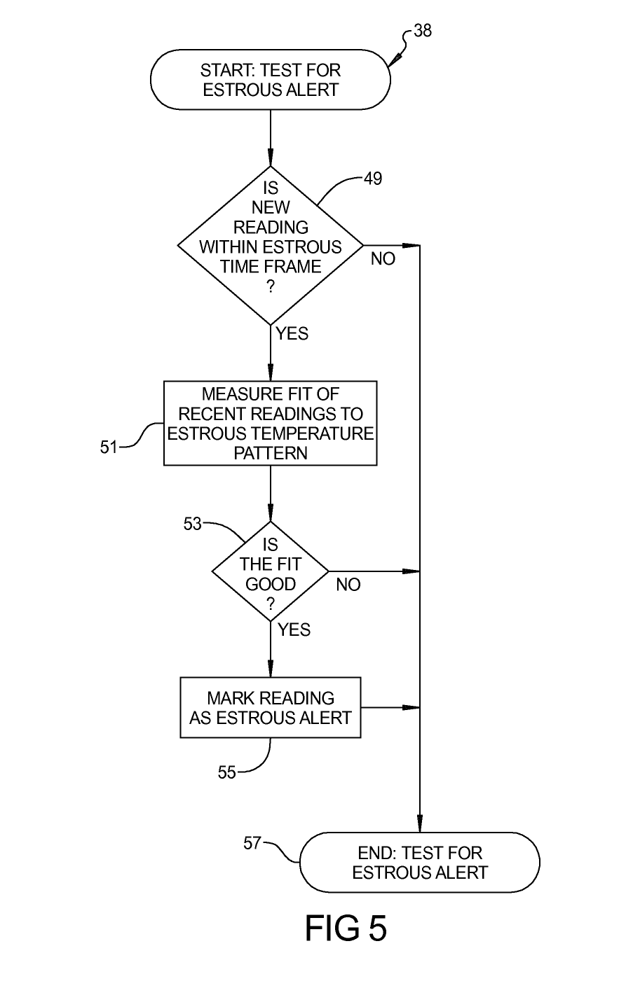

As shown in FIG. 5, an estrous alert may be generated with reliable readings. In an algorithm 38, other biological events may be predicted by temperature variations or patterns of variations from a baseline, or by signal processing calculations, correlations or convolutions, that may receive inputs from a timeframe of events or biological treatments. As shown at 51, the read data may compare a recent time period of readings to recognize a pattern of variation that coincides with additional correlations that may be made at 53, to confirm that the matching of patterns at 51 is reliable. Then, the last reading may initiate an estrous alert that may be marked for delivery with that reading of animal data records at 55 and subsequent reports of data to or from the base station.

The software may analyze sensor data, such as temperature data, along with an animal's breeding information, and events recorded by data available from medical, herd management systems, and new data sources to find a specific decreasing or changing temperature pattern determined to be a trigger of threshold. Quantification of the discovered pattern will allow the software to provide a predicted time, or time window of parturition, along with an associated probability that may be used in the presentation of information to the user, to improve the number of days in milk or maximize impregnation potential of the animals or while minimizing the costs associated with feeding and treatments inducing pregnancy.

Although the description references ruminant animals and cows in particular, such references may be read to refer to “mammals” or “ruminant animals” as applicable. For instance, in the case of animals such as dogs and cats, a different temperature measuring device could be required. However, the methods of assessment to identify differences and/or patterns could be the same.

A Sample Baseline calculation may be simple, such as an average, but a default setting or other setting with noise filter compensation may include any of the following baseline determination methods, depending on the purpose of the baseline. A DFT Baseline permits a user selectable number of days. This baseline method may first use linear interpolation to convert the bolus readings for example, temperature readings, to 72 discrete, uniform reading samples per day. The system may use a Fourier Transform to convert the time-domain readings to frequency-domain values by determining a set of coefficients to a series of scaled functions that, when summed, represent the original time-domain readings. For example, the a Discrete Fourier Transform (DFT):

$$f ( x ) = {a}_{0} + {\sum }_{n = 1}^{N} ( {a}_{n} cos \frac{n π x}{L} + {b}_{n} sin \frac{n π x}{L} )$$

may be used. This may be calculated using a Fast Fourier Transform (FFT), an optimized method of calculating the DFT. Then all frequency-domain values are set to zero except those occurring near the 1 cycle-per-day frequency. The resulting signal is then extrapolated to the time of the reading just received to determine the baseline value. This provides a filter that keeps diurnally-varying temperatures and filters out other temperatures (i.e. noise) to find a baseline rhythm following the diurnal rhythm of the individual animal.

A Band-pass Baseline uses convolution to apply both a high-pass filter and a low-pass filter (a band-pass filter). The formula used to apply a filter using convolution may be:

$$f ( {x}_{0 - N} ) = {\sum }_{n = 0}^{N} ( {a}_{n} \cdot {b}_{N - n} )$$

where a is the set of reading samples and b is the filter. The filtered value would then be used as the baseline. The convolution may be applied by calculating the dot product of the set of reading samples and the time-reversed filter samples. The filtered value would then be used as the baseline.

A Window Baseline filter compares the current temperature to previous temperatures around the same time of day over a user selectable previous number of days. First we would assign a weight to previous readings based on the time of day. Readings near the same time of day of the current temperature may be assigned a weight near one and other readings may be assigned a weight near zero. The weights may be calculated using a periodic function or a step function derived from the difference in the time of day between the current reading and reading X. A weighted average of the previous readings would then be calculated to determine the baseline value.

For example, formulas that may be used to calculate the weights could be:

$$Smooth weighting \text{:} w ( x ) = 0.1 + 0.9 \cdot {cos}^{10} ( 2 π \cdot Δ t ) ;$$

$$Square 4 \text{-} hour weighting \text{:} w ( x ) = \{ \begin{matrix}0.9 , & 0 \leq \vert Δ t mod 1 \vert \leq \frac{2}{24} \\ 0.1 , & \frac{2}{24} < \vert Δ t mod 1 \vert < \frac{22}{24} \\ 0.9 , & \frac{22}{24} \leq \vert Δ t mod 1 \vert \leq 1\end{matrix} ; or \\ Square 6 \text{-} hour weighting \text{:} w ( x ) = \{ \begin{matrix}0.9 , & 0 \leq \vert Δ t mod 1 \vert \leq \frac{3}{24} \\ 0.1 , & \frac{3}{24} < \vert Δ t mod 1 \vert < \frac{21}{24} \\ 0.9 , & \frac{21}{24} \leq \vert Δ t mod 1 \vert \leq 1\end{matrix}$$

where Δt is the difference in the time of day between the current reading and reading X measured in fractions of a day. A weighted average of the previous readings would then be calculated to determine the baseline value.

Other filters may include a standard moving average, a daily average, or other averages.

A Disease Detection assessment may be improved by the system as the assessing includes the function of comparing the animal data record of read temperature to a baseline value and, given that difference, signal disease when that difference falls outside a threshold parameter. Disease may also be signaled by directly comparing the measurements temperatures to static threshold temperatures indicating the expected absolute temperature of an animal. Note that the latter method would not account for the dynamic differences in and among the animals, whereas the improved filtering detection method would. A lack of water intake can also indicate an illness condition.

An Ovulation Detection assessment may be improved by the system as the assessing function may use correlation to find a pattern of temperatures or variations that are identifiable as an indication of biological ovulation. Correlations use previous reproduction information as a starting point. Correlation can be calculated as the dot product of the set of reading samples and the search pattern. We would then look for a peak in the correlation of a specific magnitude, and then compare the temperature at that point to the baseline to determine if ovulation has occurred. We may also incorporate the magnitude of the signal at that point, standard deviation, time of day, or other parameters into the determination. For example, a formula used to calculate a correlation plot is:

$$f ( x ) = {\sum }_{n = 0}^{N} ( {a}_{n} \cdot {b}_{x} )$$

where a is the set of reading samples and b is the search pattern. We would then look for a peak in f(x) of a specific magnitude, and then compare the temperature at that point to the baseline to determine if ovulation has occurred. We may also incorporate the magnitude of the signal at that point, standard deviation, time of day, or other parameters into the determination.

A Parturition Detection assessment by the system may be the same as the method for ovulation detection, or include a differently defined baseline calculation, or a refined assessment for creating an alert.

A Water Effect Detection for filtering noise in the TempTrack® system may be used to handle instances where the animal drinks water, thus affecting the ruminal temperature. The method used to locate and handle “water effect” readings may include some or all of the following: Locate a “water effect” reading by using a correlation with a drinking event or a sequence of readings to find a water effect pattern. This may be similar to the use of correlation in Ovulation Detection; alternatively, calculating the slope between two readings, t₋₁ and t₀. For example, if slope s is below a certain threshold (e.g. −1° C./hour), t₀ would be marked as a “water effect” reading.

The system may also modify animal data records of the read data by removing that reading from calculations, or measuring the magnitude of the drop in temperature and, based on the magnitude, calculating a “water effect duration” using a certain factor (e.g. 1 hour/° C.), then removing that reading and any reading that occurs within the “water effect duration” after that reading from calculations. Alternatively, the system may measure the slope between that reading and the following reading and, with the magnitude of the drop in temperature or other parameters, calculate an estimated “correction factor” to be added to that reading. Any calculations that may use water effect detection could then calculate a baseline, detect ovulation or detect other biological conditions using the post-modified data as animal data records.

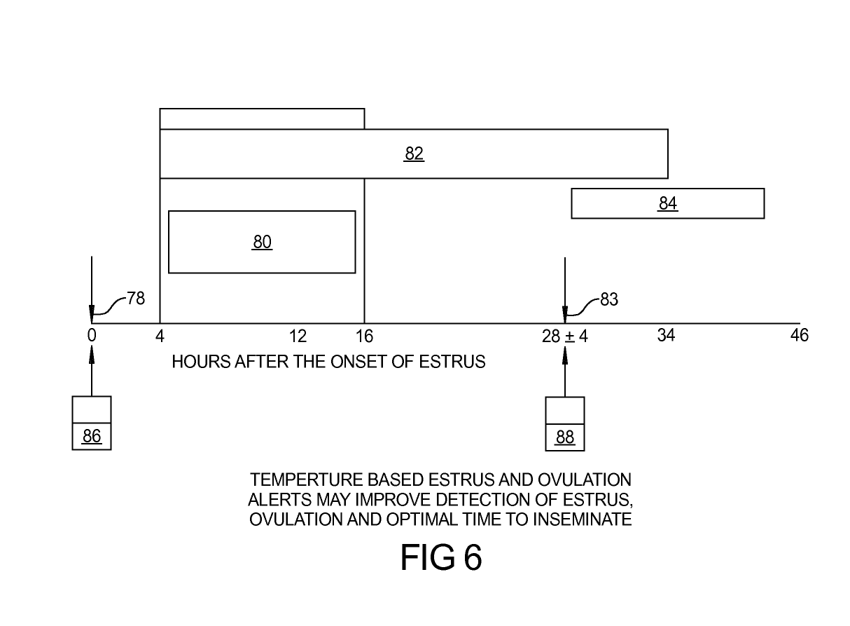

As shown in FIG. 6, a timeline of biological events associated with ovulation may provide an advance alert to start insemination during an optimal time period. The optimal time to start would be after a voluntary waiting period (VWP), that enables the cow to recover fully to maximize milk production, avoid long term reduced milk production, and improve receptiveness to insemination. As shown at 80, an optimal time for effective insemination is about 4 hours from the onset of standing estrous 78 until about 16 hours from the onset of standing estrous 78. The sperm remain viable for about 24-34 hours as demonstrated at 82. Ovulation 83 may be expected to release an ova that remains viable about 6-12 hours as shown at 84. As a result, the system's assessment provides alerts 86 for early detection or prediction of the onset of estrous and alert 88 for detection or prediction of ovulation that improve management of the herd. The system reduces missed or untimely impregnation that results in open days. Open days occur during the period in which the cows are not impregnated but must be fed and cared for without extending milk production or birth opportunity. As a result, assessments of temperature readings as animal data records have proven effective for improving cost reduction and effective management of the herd by detecting estrous, anestrous, and silent estrous, ovulation, optimal time for impregnation, and conditions of animals in the herd.

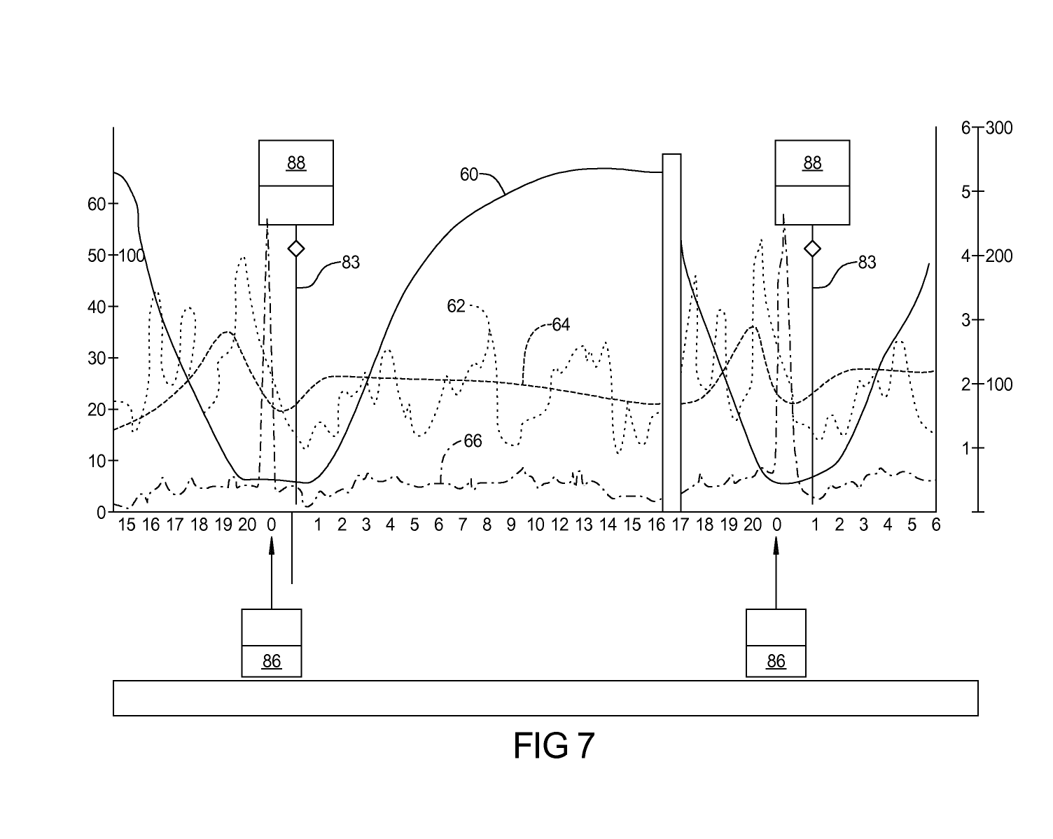

Referring now to FIG. 7, monitoring of the bovine estrous cycle using an embodiment of the invention demonstrates that sensed temperature data correlates to breeding cycle events as temperature readings are assessed by processing animal data records. Such processing may correlate temperature with summarized levels of luteinizing hormones (LH—66), progesterone (PRO—60), follicle stimulating hormones (FSH—62) and estrogen (EST—64), identifying estrus (86), predicting ovulation (88) and confirming ovulation.

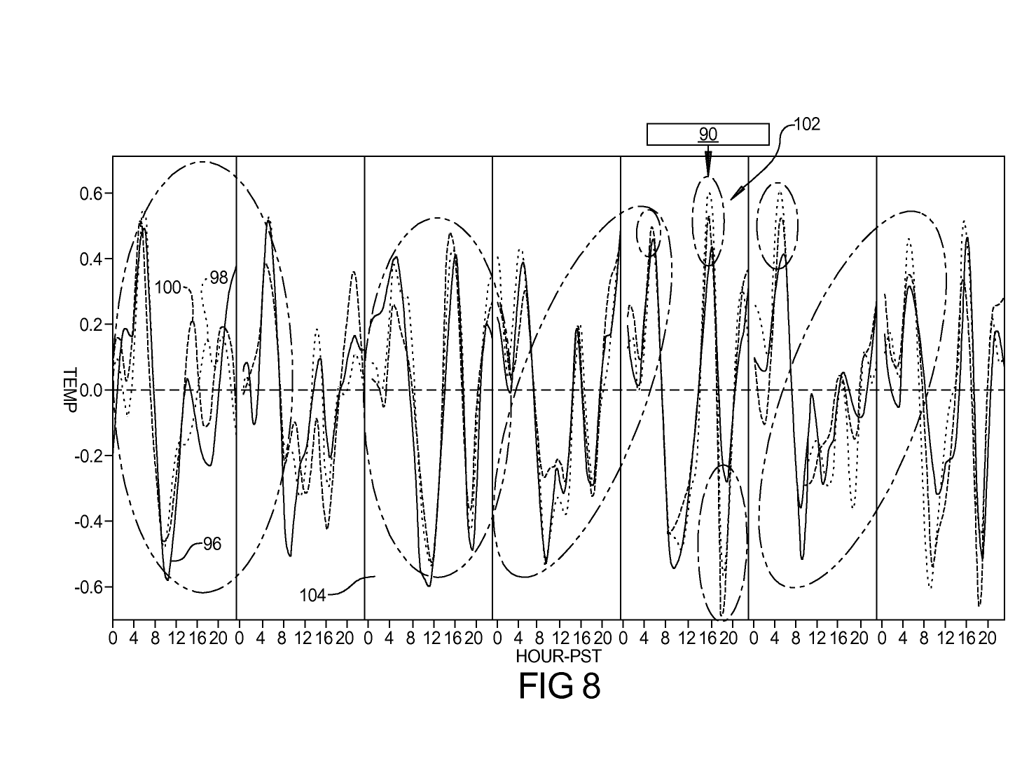

As shown in FIG. 8, a summary chart showing temperature detections and variations from a baseline over time, as processed to identify estrus, predict ovulation and confirm ovulation by variations from a baseline. A plot 96 designates cows not pregnant, no ovulation per ultrasound tests and no ovulation per blood progesterone tests; 98 designates cows not pregnant, ovulated per ultrasound tests and ovulated per blood progesterone tests; 100 designates cows pregnant, ovulated per ultrasound tests and ovulated per blood progesterone tests.

The small ovals below 90 show a temperature correlation with animals that 1.) did not ovulate per ultrasound and blood progesterone tests and did not become pregnant, 2.) did ovulate per ultrasound and blood progesterone tests and did not become pregnant and 3.) did ovulate per ultrasound and blood progesterone tests and did become pregnant. The large oval to the left of 90 shows temperature pattern from left to right temperature increasing pattern leading to ovulation.

The comparison of the detected and assessed animal data records with known “gold standards” of health monitoring such as ultrasound and blood progesterone confirm the ovulation indication 90 identified by the assessed data records. Similarly, separate ovulation confirmation by ultrasound or blood progesterone is represented. The large elliptical circles 104 represent unique temperature pattern where the third oval from the left shows a change in temperature patterns that indicate where an ovulation indication could occur.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

What is claimed is:

1. A method for obtaining early detection of biological events in individual animals of a herd comprising:
sensing core body temperature in the individual animal autonomously;
identifying animal data records of each temperature sensing;
accumulating a selected set of animal data records;
assessing the selected set of identifying records for changes indicative of imminent biological events; and
autonomously reporting results of the assessing.

2. The method as defined in claim 1 wherein accumulating includes compensating for interference factors in selecting the set of animal data records.

3. The method as defined in claim 2 wherein the compensating includes establishing at least one threshold range for accepting the identifying data record for accumulating in the selected set.

4. The method as defined in claim 3 wherein the threshold relates to a predetermined temperature range of the core body temperature.

5. The method as defined in claim 3 wherein the threshold is a pattern of temperature variations for a selected period of the accumulating selected set of identifying data records.

6. The method as defined in claim 5 wherein the threshold is a pattern of temperature variations for a selected sequence of periods of the accumulating selected set of animal data records.

7. The method as defined in claim 3 wherein the compensating includes establishing a temperature baseline from a selected accumulation of identifying animal data records.

8. The method as defined in claim 3 wherein the compensating comprises processing an algorithm that establishes a temperature baseline for an individual animal.

9. The method as defined in claim 3 wherein said processing includes using a Fourier Transform to convert time-domain readings to frequency-domain values by determining a set of coefficients to a series of scaled functions that, when summed, represent the original time-domain readings.

10. The method as defined in claim 3 wherein said establishing comprises filtering to keep diurnally-varying temperatures and filter out other temperatures to find a baseline rhythm following the diurnal rhythm.

11. The method as defined in claim 3 wherein establishing comprises applying both a high-pass filter and a low-pass filter (a band-pass filter).

12. The method as defined in claim 11 wherein establishing applies a filter using convolution.

13. The method as defined in claim 8 wherein a window baseline compares the current temperature reading to previous temperature readings around same time of day over a user selectable previous number of days.

14. The method as defined in claim 13 wherein same-time-of-day readings are weighted by a periodic weighting factor that varies by time of day.

15. The method as defined in claim 7 wherein establishing the baseline includes selecting a set of identifying animal data records.

16. The method as defined in claim 15 wherein the selecting comprises compensating for temperature variations based on water effect.

17. The method as defined in claim 16 comprising using correlating to find a water effect pattern.

18. The method as defined in claim 16 comprising calculating slope between two readings.

19. The method as defined in claim 16 comprising adjusting a reading from calculations, if it meets a threshold difference from baseline.

20. The method as defined in claim 16 comprising measuring the magnitude of the drop in temperature and, based on the magnitude, calculating a “water effect duration” using a timing factor of x hour/° C.

21. The method as defined in claim 16 comprising measuring slope between readings and calculating an estimated correction factor based on the magnitude of the drop.

22. The method as defined in claim 8 wherein the assessing comprises monitoring variations of read animal data records from the baseline.

23. The method as defined in claim 22 wherein the assessing comprises monitoring patterns of variations of animal data records from the baseline.

24. The method as defined in claim 1 wherein the assessing includes performing signal processing techniques for changes in temperature animal data records identified as representative of imminent biological events.

25. The method as defined in claim 8 wherein the assessing includes performing signal processing techniques on animal data records as representative of imminent biological events related to variations of animal data records from the baseline.

26. A system for generating early detection of biological events in an animal comprising:
a sensor for detecting core body temperatures autonomously and transmitting animal data records correlated to each temperature sensing;
a receiver receiving and identifying animal data records from the receiver;
a basestation accumulating and identifying data records from the receiver and transmitting these records to a processor;
a processor for performing algorithms assessing the selected set of animal data records for temperature changes indicative of imminent biological events; and
a processor for communicating autonomously reporting results of the assessing, and prognosticating an alert about an expected biological event for the animal generating the identifying data records.

27. The system as defined in claim 26 wherein said communicator prognosticates an illness.

28. The system as defined in claim 26 wherein said communicator prognosticates an estrus period.

29. The system as defined in claim 26 wherein said communicator prognosticates an ovulation event.

30. The system as defined in claim 26 wherein said communicator prognosticates a parturition event.
